(19) United States
(12) Patent Application Publication
(10) Pub. No.: US 2006/0105342 A1
(43) Pub. Date: May 18, 2006

(54) Computerized systems for formation and update of databases

(76) Inventors: Mario Villena, Miami, FL (US); Jose Villena, Miami, FL (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 10/536,692

(22) PCT Filed: Aug. 31, 2004
(86) PCT No.: PCT/US04/28217

§ 371 Date: Sep. 28, 2005

(51) Int. Cl. C12Q 1/70; C12Q 1/68

(52) U.S. Cl. 435/6; 435/5

(57) ABSTRACT

An apparatus for creating and maintaining a database containing information relating to residential properties includes an AVM device configured to receive information relating to residential properties, producing AVM values for each property and updating an AVM database with the AVM values.

### FIELD OF THE INVENTION

This disclosure relates to a computer-based system for creating and maintaining massive databases containing computationally complex and novel property information.

### BACKGROUND OF THE INVENTION

Real estate is a multi-billion dollar industry touching virtually every strata of our society. Although the industry has consolidated somewhat during the last ten years, it remains highly fragmented. Unfortunately, the “power to transact” is largely concentrated in the hands of real estate brokers and agents regardless of the fact that entry and exit barriers are low. Further, the range of available buying and selling options are limited since the services provided by practically every real estate professional are virtually the same throughout the entirety of the United States and beyond.

Unfortunately, the real-estate industry has been highly resistant to the threat of new and potentially disruptive technologies as consumer behavior have been almost completely ignored. While a number of new computer-based tools are under development or have been proposed, their effect has either been inconsequential or specifically designed to preserve the status quo of the real estate industry, rather than directed to transform the industry to the benefit of property owners. Accordingly, new technological approaches relating to real estate are desirable.

### SUMMARY OF THE INVENTION

In one aspect, a storage medium containing a database of property related information that, when accessed by a computer, can enable a user to perform a number of property-related queries includes records on substantially every residential property in a defined geographic region. Each entry of the property database can itself include an identifying field identifying a specific property and an automated valuation field containing an AVM produced value of the identified property.

In another aspect, an apparatus for creating and maintaining a database containing information relating to residential properties includes an AVM device configured to receive information relating to substantially every known property within a defined geographical region that is currently offered for sale, further configured to perform an AVM operation on each property to produce an AVM value for each property and still further configured to create an AVM database containing entries of a plurality of the AVM values.

There has thus been outlined, rather broadly, certain embodiments of the invention in order that the detailed description thereof herein may be better understood, and in order that the present contribution to the art may be better appreciated. There are, of course, additional embodiments of the invention that will be described or referred to below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 depicts a networked system capable of allowing users to access AVM technology.

FIG. 2 is a block diagram of a system capable of performing AVM-related services.

FIG. 3 depicts various geographic regions of interest.

FIG. 4 depicts AVM database information.

FIG. 5 is a flowchart outlining an exemplary operation for performing AVM-related services.

FIG. 6 is a flowchart outlining a second exemplary operation for performing AVM-related services.

### DETAILED DESCRIPTION

Automated Valuation Methodology (AVM) is a computer-based technology that has been used to determine the market value of real estate for nearly a decade. Unfortunately, AVM technology is very expensive. As a result, the available AVM providers are generally limited to a very few large corporations. The downside of having such limited pool of AVM providers keeps the price extremely high and the availability extremely limited. For example, one web-based service will provide an AVM valuation of a specific residential property for $25. While this may be appropriate if one desires to look at a limited number of properties or determine a mortgage limit for a specified property, its utility as a market analysis tool to the small investor is practically nil as performing a comparative analysis of properties in even a small geographic can cost tens of thousands of dollars.

Another downfall of the limited availability of AVM providers is quality. In an study of four AVMs, Standard & Poor's found that, while known AVM can (in many circumstances) provide decent property estimates, known AVM systems are documented as over-estimating property values by as much as two-hundred percent. As a result of the spotty nature of AVMs, lenders are forced to continue to rely heavily upon appraisers, who in turn rely on what is known as the “Appraisal Institute Residential Database” (AIRD). As the AIRD is basically of use only to professional appraisers and only for specific identified properties, its usefulness as a marketing tool to the small investor is also practically nil due to the high expense of the human element.

In view of these circumstances, the inventors of the disclosed methods and systems have created an entirely new approach to empower buyers and sellers of real estate property. Against industry trends and traditions, the inventors have invested greatly in their own AVM technology, and applied it in unconventional and novel ways. Accordingly, the inventors have a unique standing in the relevant industry with the ability to perform massive numbers of AVM valuations at negligible costs. As a result, the inventors have created novel applications that can take advantage of the strengths of AVM technology while limiting their known liabilities. For example, by employing AVM technology not against various specific properties, but against practically every property in a given geographical region, entirely new approaches for investing can be formulated with negligible costs and before any substantial money is ever invested by a given consumer.

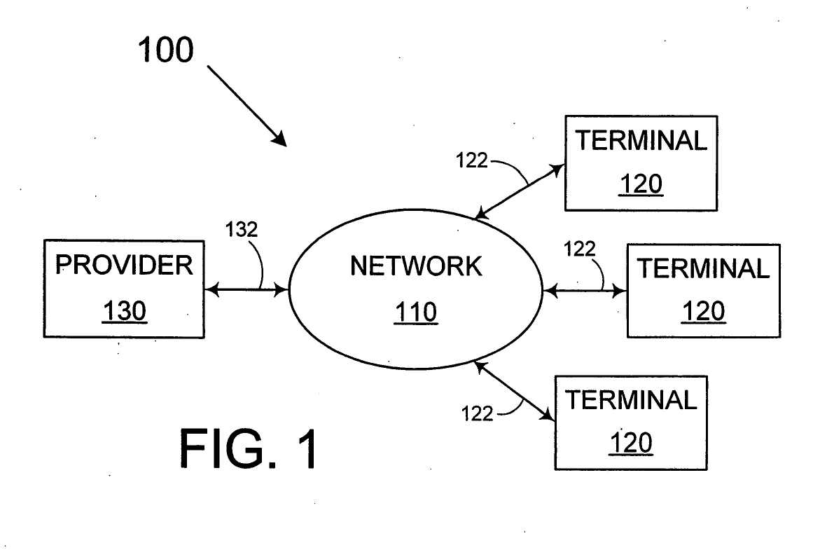

FIG. 1 depicts an exemplary networked-system 100 configured to enable individual consumers/investors to effectively utilize AVM technology. As shown in FIG. 1, the networked-system 100 includes a provider 130 coupled to a network 110 via link 132, as well as a number of terminals 120 coupled to the network 100 via respective links 122.

In operation, the provider 130 can first perform a number of set-up operations, such as creating and maintaining a database of all (or substantially all) known properties in a particular geographical region, performing an AVM valuation of such properties and identifying all such properties that are offered for sale. Once the appropriate information is amassed and prepared, the provider 130 can service anyone who may desire to employ any of a number of property-related services made available by the provider 130. In the present embodiment, such services can be accessed using any of the available terminals 120.

The terminals 120 of the immediate example are personal computers capable of interfacing with a network. However, in various embodiments the terminals 120 can include any of a variety of communication devices, such as personal computers, PDAs, telephones and cell-phones (with and without graphic displays), television sets with special two-way interfaces or any other known or later-developed communication device capable of communicating with an automated service provider without departing from the spirit and scope of the present disclosure.

The exemplary provider 130 is a computer-based server capable of accessing the Internet. However, as with the terminals 120, it should be appreciated that the provider 130 can take any number of forms, such as a server, a personal computer, a mainframe and so on.

The exemplary network 110 is a publicly available portion of the Internet. However, in other embodiments the network 110 can be any viable combination of devices and systems capable of linking computer-based systems including a wide area network, a local area network, a connection over an intranet or extranet, a connection over any number of distributed processing networks or systems, a virtual private network, the Internet, a private network, a public network, a value-added network, an intranet, an extranet, an Ethernet-based system, a Token Ring, a Fiber Distributed Datalink Interface (FDDI), an Asynchronous Transfer Mode (ATM) based system, a telephony-based system including T1 and E1 devices, a wired system, an optical system, a wireless system and so on.

The various links 122 and 132 of the present embodiment are a combination of devices and software/firmware configured to couple computer-based systems to the Internet over a wired line. However, it should be appreciated that, in differing embodiments, the links 122 and 132 can take the forms of modems, networks interface card, serial buses, parallel busses, WAN or LAN interfaces, wireless or optical interfaces and the like as may be desired or otherwise dictated by design choice.

Returning to FIG. 1, once the provider 130 has performed the above-mentioned initial tasks, the provider 130 can then provide a variety of services to potential users via the terminals 120. In order to service such users and enable various features, the provider 130 of the present embodiment can provide a number of web-pages formatted using HTML, XML, Flash or any other viable publishing standard, such that users accessing the web-pages can do so using nothing more than a commercially available web-browser. However, it also should be appreciated that the provider 130 can also use any number of standardized or specially designed software packages as may be necessary or otherwise desired under a given set of particular circumstances. For example, users accessing the provider 130 using a telephone might use a VoiceXML interface, users accessing the provider 130 using a PDA or FAX-based interface might use a custom program and so on.

A first available service provided by the provider 130 includes the capacity to allow users to query its databases to identify and list all real properties in a given geographic region (such as a region defined by a zip-code, a state, city or county, a school zone, a housing development etc) as well as identify all known properties offered for sale in such region. The provider 130 can further perform queries to identify different types of properties (e.g., single-family dwellings, townhouses, condos, duplexes etc), identify properties based on sale price, tax valuation, number of bedrooms, number of bathrooms, acreage or any other aspect of a property that a consumer/investor might care about and that can be described on a computer medium.

Additionally, the provider 130 can perform queries and identify properties based on AVM valuation. For example, given that the provider 130 can access a database of substantially all known properties in a particular city, and given that the database contains an AVM value for each property, the provider 130 could identify all properties within the city having an AVM value between $150,000 and $100,000, identify all townhouses having an AVM value between $150,000 and $100,000 and so on.

Still further, the provider 130 can perform a “Differential Valuation Search” (DVS) to identify properties based on both their respective AVM values and sale prices. That is, a particular user may wish to identify various properties in a particular region that are offered for sale at a price substantially below their AVM values. For example, by formulating a query to include a preferred school district and an interest in townhouses that are offered for sale at a price at least 20% below their respective AVM values, the provider 130 can appropriately respond and identify any such properties with but a short wait and modest fee.

In the present embodiment, the provider 130 can identify such properties by issuing a literal list of such properties in any number of ordered ways, e.g., ascending/descending sale prices, ascending/descending AVM values, ascending/descending DVS values etc. In other embodiments, however, results can be reported using any combination of lists, graphics (e.g., maps), voiced responses (using, for example, VoiceXML technology) and so on.

In addition of a percentage-based DVS query, the provider 130 can also perform DVS queries based on the absolute difference in sale price and AVM value. Still further, DVS queries can be formed based on a “modified absolute difference” in sale price and AVM value, i.e., the absolute difference discounting various financial factors, such as condo fees, insurance rates, tax assessments, reported utility rates or any other known or later acknowledged item that can affect the investment value of a property. For instance, while a particular user may wish to identify all single-family dwellings in a city that are for sale for at least $10,000 below their AVM value, the user may desire to discount, change the ordering of, highlight or completely eliminate properties that might pass the differential valuation requirement but are encumbered by housing association fees, unusual insurance requirements, reside in high-crime neighborhoods and so on.

Another optional feature of the provider 130 is its ability to perform AVM or DVS searches on dissimilar properties to compare unlike properties for value. For example, a user may wish to identify all condos of a specific type (e.g., 2 bedrooms, 2 bathrooms and 1200+ sq ft) in a specific price range with a DVS of 20% simultaneously with all single-family dwellings of a given DVS (or AVM) range having at least three bedrooms and located on at least two acres. By allowing such complex queries, the provider 130 can issue important financial data to investors thus allowing them to compare and contrast various investment opportunities, as well as present attractive options to potential buyers more interested in living in one of several types of (well-priced) properties than investing merely for profit.

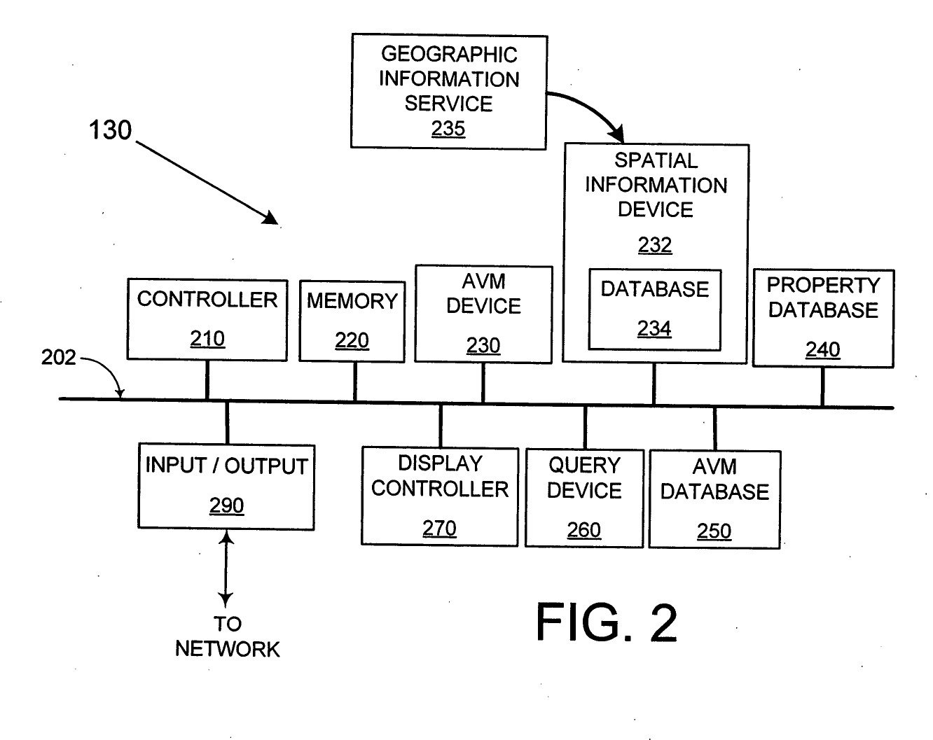

FIG. 2 is an exemplary provider 130 capable of providing a variety of property related services including services that use AVM technology. As shown in FIG. 2, the exemplary provider 130 includes a controller 210, a memory 220, an AVM device 230, a spatial information device 232 (with optional spatial database 234) supported by a Geographical Information Service (GIS) 235, a property database 240, an AVM database 250, a query device 260, a display controller 270 and an input/output device 290. The above components 210-290 are coupled together by control/data bus 202.

Although the exemplary provider 130 of FIG. 2 uses a bussed architecture, it should be appreciated that any other architecture may be used as is well known to those of ordinary skill in the art. For example, in various embodiments, the various components 210-290 can take the form of separate electronic components coupled together via a series of separate busses.

Still further, in other embodiments, one or more of the various components 210-290 can take form of separate servers coupled together via one or more networks. Additionally, it should be appreciated that each of components 210-290 advantageously can be realized using multiple computing devices employed in a cooperative fashion. For example, by employing two or more separate computing devices, e.g., servers, to provide spatial information for each computing device used to make AVM calculations, a processing bottleneck can be reduced/eliminated and the overall computing time to produce AVM valuations and other services can be drastically reduced.

It also should be appreciated that some of the above-listed components can take the form of software/firmware routines residing in memory 220 and be capable of being executed by the controller 210, or even software/firmware routines residing in separate memories in separate servers/computers being executed by different controllers. Further, it should be understood that the functions of any or all of components 230-270 can be accomplished using object-oriented software, thus increasing portability, software stability and a host of other advantages not available with non-object-oriented software.

In operation, the provider 130 can first perform a number of setup operations including populating the property database 240 with information about every property within a geographic region of interest as well as identify which properties are offered for sale and the method of sale (e.g., for sale by owner, via an agent etc). While the exemplary provider 130 uses a collection of public and private records (e.g., MLS databases, secondary databases, tax databases, newspaper ads and ads placed specifically with the provider 130) the particular sources of information can vary as required or otherwise found advantageous.

Once the property database 240 is populated, the AVM device 230, under control of the controller 210, can perform an AVM valuation on each property in the property database 240. The exemplary AVM device 230 is based on a combination of heuristic and statistical technologies. However, it should be appreciated that the particular form and functionality of the AVM device 230 can vary from embodiment to embodiment as the technology evolves or as otherwise can be found advantages in various circumstances.

In order to support the AVM device 230, the exemplary provider 130 employs its spatial information device 232 to provide high-resolution spatial data for the various properties of interest, such as high-resolution spatial data including absolute position data, relative position data (e.g., from one property to another), relative direction data etc.

The exemplary spatial information device 232 provides an advantage over conventional AVM systems in that the inventors of the present methods and systems have found a way to reliably and consistently provide absolute and relative spatial information measured in increments of feet (or meters). This is in stark contrast to conventional AVM systems which can only provide distance resolutions literally measured in miles and having negligible, if any, directional information. This is because unlike the exemplary GIS 235, which derives position information using GPS global positioning data to perform high-resolution surveys, conventional systems rely on position approximations based on township, range, section and subdivision information that may or may not be contained in property databases.

While the exemplary spatial information device 232 can provide spatial resolution down to a meter or less, it should be appreciated that more coarse resolutions may be employed with varying degrees of performance. For example, by using a resolution of ten meters, one-hundred meters, two-hundred meters, five-hundred meters and even a kilometer AVM valuation accuracies may be expected to degrade, but may still provide better performance than conventional techniques due to superior resolution, consistency and/or the availability of vector information.

While GIS is a known technology used for land use planning, transportation planning, environmental management and other uses, the exemplary methods and systems are unique in that there are no known instances where a GIS has ever been used for any form of property valuation whether it be by AVM or other means. Similarly, there are no known instances where any form of survey data (by GIS, GPS or other means) has ever been used for any form of property valuation.

In various embodiments, a Geographic Information System can be a combination of computer hardware, software, personnel, survey equipment and data that can enable one to do one or more of store, create, and analyze spatial data. Spatial data can be any information that is referenced to a location. In short, a GIS can be more than a map in that it can hold an underlying database. In order to assure consistent, reliable high-resolution spatial data, the exemplary GIS 235 derives spatial positioning information using survey data, such as survey data derived in part from GPS equipment.

By incorporating a GIS into the exemplary provider 130, a plethora of advantages over other systems are gained, including the availability of a visual representation of the geographic region under analysis. For example, the GIS of the exemplary embodiment can provide a map-like display of objects, such as parcels, schools, police stations, fire hydrants, churches etc. Another advantage to using a GIS is that the above map-like representation is more than a collection of spatially distributed symbols as each symbol/icon has a variety of information associated with it. For example, by referencing a particular “house” symbol, an operator can pull up a host of (1) geographic information, such as latitude, longitude, elevation, county, school district etc, (2) structural information, such as acreage, age, number of bedrooms etc, and various miscellaneous items of information, such as sale history, mortgage etc.

Another advantage of the exemplary system is that it has the ability to highlight on a map the comparables selected for a given target property, and the ability to highlight on a map the comparables selected for AVM valuation.

Still another advantage of the present GIS system is its ability to provide distance calculations down to meters/feet between any two objects as well as directional information. The fine resolution of distances and/or direction can provide an incredible advantage in that, the closer that two structurally comparable houses are, the more likely the sale price of one property will reflect on the value of the other property. Accordingly, the exemplary GIS system can make it possible to weight various sale values as a function of distance in a way that was never possible before.

Additionally, by using true spatial vector information (distance plus angle (or other coordinate system)), AVM calculations can be further refined. For example, if a particular house is structurally comparable to four other houses, and the four houses have values that vary (1) as a function of how far west each house lies and (2) as a function of their proximity to a river or train station, it should be appreciated that high-resolution distance information and angular information may be valuable.

Although the use of GIS (or equivalent spatial) information can be invaluable, processing such information can be relatively expensive in terms of the computation power required to derive accurate AVM valuations via GIS information. As making computationally expensive determinations is rarely acceptable in a web environment, the present AVM system overcomes this problem by pre-processing AVM valuations, preprocessing spatial information and using multiple computer systems to alleviate processing bottlenecks, then allowing users to access some or all of the preprocessed data.

Returning to FIG. 2, as each property in the property database 240 is processed and an AVM value is determined, the AVM device 230 can place the AVM data, along with other data of interest, into the AVM database 250. In the present embodiment, the exemplary AVM device 230 can update the AVM database 250 often and, in some embodiments, can update the AVM database after every sale of a property.

For example, in a particular embodiment, the controller 210 can update the property database 240 to signal that a townhouse in a particular neighborhood recently sold for a given amount of money along with other useful information about the transaction, such as concessions made by the buyer or seller (e.g., points paid by seller) that might better reflect the actual sale value of the property. The AVM device 230 can then subsequently update the AVM values of properties that might be affected by the sale, such as comparable townhouses and other properties in the immediate area, then update the AVM database 250 appropriately.

FIG. 2 is an exemplary provider 130 capable of providing a variety of property related services including services that use AVM technology. As shown in FIG. 2, the exemplary provider 130 includes a controller 210, a memory 220, an AVM device 230, a spatial information device 232 (with optional spatial database 234) supported by a Geographical Information Service (GIS) 235, a property database 240, an AVM database 250, a query device 260, a display controller 270 and an input/output device 290. The above components 210-290 are coupled together by control/data bus 202.

Although the exemplary provider 130 of FIG. 2 uses a bussed architecture, it should be appreciated that any other architecture may be used as is well known to those of ordinary skill in the art. For example, in various embodiments, the various components 210 290 can take the form of separate electronic components coupled together via a series of separate busses.

Still further, in other embodiments, one or more of the various components 210-290 can take form of separate servers coupled together via one or more networks. Additionally, it should be appreciated that each of components 210-290 advantageously can be realized using multiple computing devices employed in a cooperative fashion. For example, by employing two or more separate computing devices, e.g., servers, to provide spatial information for each computing device used to make AVM calculations, a processing bottleneck can be reduced/eliminated and the overall computing time to produce AVM valuations and other services can be drastically reduced.

It also should be appreciated that some of the above-listed components can take the form of software/firmware routines residing in memory 220 and be capable of being executed by the controller 210, or even software/firmware routines residing in separate memories in separate servers/computers being executed by different controllers. Further, it should be understood that the functions of any or all of components 230-270 can be accomplished using object-oriented software, thus increasing portability, software stability and a host of other advantages not available with non-object-oriented software.

In operation, the provider 130 can first perform a number of setup operations including populating the property database 240 with information about every property within a geographic region of interest as well as identify which properties are offered for sale and the method of sale (e.g., for sale by owner, via an agent etc). While the exemplary provider 130 uses a collection of public and private records (e.g., MLS databases, secondary databases, tax databases, newspaper ads and ads placed specifically with the provider 130) the particular sources of information can vary as required or otherwise found advantageous.

Once the property database 240 is populated, the AVM device 230, under control of the controller 210, can perform an AVM valuation on each property in the property database 240. The exemplary AVM device 230 is based on a combination of heuristic and statistical technologies. However, it should be appreciated that the particular form and functionality of the AVM device 230 can vary from embodiment to embodiment as the technology evolves or as otherwise can be found advantages in various circumstances.

In order to support the AVM device 230, the exemplary provider 130 employs its spatial information device 232 to provide high-resolution spatial data for the various properties of interest, such as high-resolution spatial data including absolute position data, relative position data (e.g., from one property to another), relative direction data etc.

The exemplary spatial information device 232 provides an advantage over conventional AVM systems in that the inventors of the present methods and systems have found a way to reliably and consistently provide absolute and relative spatial information measured in increments of feet (or meters). This is in stark contrast to conventional AVM systems which can only provide distance resolutions literally measured in miles and having negligible, if any, directional information. This is because unlike the exemplary GIS 235, which derives position information using GPS global positioning data to perform high-resolution surveys, conventional systems rely on position approximations based on township, range, section and subdivision information that may or may not be contained in property databases.

While the exemplary spatial information device 232 can provide spatial resolution down to a meter or less, it should be appreciated that more coarse resolutions may be employed with varying degrees of performance. For example, by using a resolution of ten meters, one-hundred meters, two-hundred meters, five-hundred meters and even a kilometer AVM valuation accuracies may be expected to degrade, but may still provide better performance than conventional techniques due to superior resolution, consistency and/or the availability of vector information.

While GIS is a known technology used for land use planning, transportation planning, environmental management and other uses, the exemplary methods and systems are unique in that there are no known instances where a GIS has ever been used for any form of property valuation whether it be by AVM or other means. Similarly, there are no known instances where any form of survey data (by GIS, GPS or other means) has ever been used for any form of property valuation.

In various embodiments, a Geographic Information System can be a combination of computer hardware, software, personnel, survey equipment and data that can enable one to do one or more of store, create, and analyze spatial data. Spatial data can be any information that is referenced to a location. In short, a GIS can be more than a map in that it can hold an underlying database. In order to assure consistent, reliable high-resolution spatial data, the exemplary GIS 235 derives spatial positioning information using survey data, such as survey data derived in part from GPS equipment.

By incorporating a GIS into the exemplary provider 130, a plethora of advantages over other systems are gained, including the availability of a visual representation of the geographic region under analysis. For example, the GIS of the exemplary embodiment can provide a map-like display of objects, such as parcels, schools, police stations, fire hydrants, churches etc. Another advantage to using a GIS is that the above map-like representation is more than a collection of spatially distributed symbols as each symbol/icon has a variety of information associated with it. For example, by referencing a particular “house” symbol, an operator can pull up a host of (1) geographic information, such as latitude, longitude, elevation, county, school district etc, (2) structural information, such as acreage, age, number of bedrooms etc, and various miscellaneous items of information, such as sale history, mortgage etc.

Another advantage of the exemplary system is that it has the ability to highlight on a map the comparables selected for a given target property, and the ability to highlight on a map the comparables selected for AVM valuation.

Still another advantage of the present GIS system is its ability to provide distance calculations down to meters/feet between any two objects as well as directional information. The fine resolution of distances and/or direction can provide an incredible advantage in that, the closer that two structurally comparable houses are, the more likely the sale price of one property will reflect on the value of the other property. Accordingly, the exemplary GIS system can make it possible to weight various sale values as a function of distance in a way that was never possible before.

Additionally, by using true spatial vector information (distance plus angle (or other coordinate system)), AVM calculations can be further refined. For example, if a particular house is structurally comparable to four other houses, and the four houses have values that vary (1) as a function of how far west each house lies and (2) as a function of their proximity to a river or train station, it should be appreciated that high-resolution distance information and angular information may be valuable.

Although the use of GIS (or equivalent spatial) information can be invaluable, processing such information can be relatively expensive in terms of the computation power required to derive accurate AVM valuations via GIS information. As making computationally expensive determinations is rarely acceptable in a web environment, the present AVM system overcomes this problem by pre-processing AVM valuations, preprocessing spatial information and using multiple computer systems to alleviate processing bottlenecks, then allowing users to access some or all of the preprocessed data.

Returning to FIG. 2, as each property in the property database 240 is processed and an AVM value is determined, the AVM device 230 can place the AVM data, along with other data of interest, into the AVM database 250. In the present embodiment, the exemplary AVM device 230 can update the AVM database 250 often and, in some embodiments, can update the AVM database after every sale of a property.

For example, in a particular embodiment, the controller 210 can update the property database 240 to signal that a townhouse in a particular neighborhood recently sold for a given amount of money along with other useful information about the transaction, such as concessions made by the buyer or seller (e.g., points paid by seller) that might better reflect the actual sale value of the property. The AVM device 230 can then subsequently update the AVM values of properties that might be affected by the sale, such as comparable townhouses, other properties in the immediate area and/or properties within a certain distance etc, then update the AVM database 250 appropriately.

Another approach to updating AVM databases includes update (automatic/periodic or by command) of an entire database. However, as an such an update process can be computationally expensive, use of multiple processing systems might be necessary in order to reduce the overall processing time.

Another approach to updating AVM databases includes update of an database for a specified region, e.g., a township or a predefined region defined by X-Y boundaries. In various embodiments, such an update can occur on command, automatically on the basis of a regular period or automatically on a basis of whenever a given number of properties (like or not) are sold within the region.

Still another approach to updating AVM databases includes automatically update of like properties, e.g., condos, within a given region with such update occurring on the basis of a regular period or on a basis of whenever a given number of properties (like or not) are sold within the region etc.

Yet another approach to updating AVM databases includes update of individual properties. A first variant of this approach is to update individual properties automatically on a regular periodic basis. For example, for a particular property put on the market, the property AVM value can be recomputed every N-number of days and optionally scheduled for early morning hours (or other low-usage periods) to alleviate computer processing bandwidth. Other variants can include update by command or a combination of automatic and command approaches.

In yet other embodiments to updating AVM databases, AVM update can occur on any combination of local market activity, predefined periods, similarity of properties, by region, by command and so on.

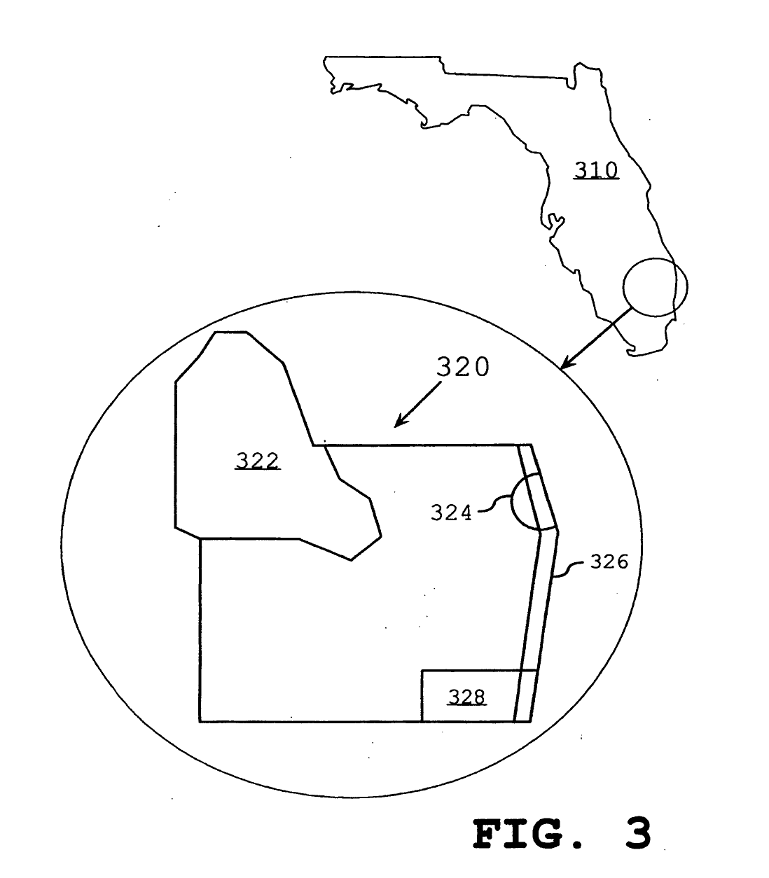

FIG. 3 depicts various types of geographical regions of interest. As shown in FIG. 3, a first region 310 (Florida) can be considered as a definable region of interest as well as a county 320 (Palm Beach). While the county may a better region to work with given its specificity, FIG. 3 shows that the county 320 can be conceptually divided into a zip-code region 322, a region of an incorporated city 324, an exemplary school district 328 or beachfront property 326. Noting that the various regions 310-328 can overlap, it should be appreciated that it can be useful to define regions by multiple, overlapping geographical attributes, e.g., beachfront property 326 found in school district 328.

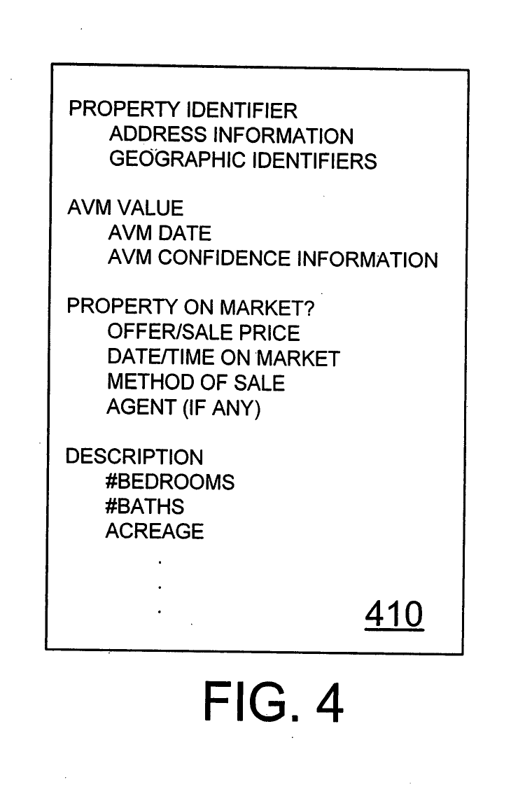

FIG. 4 depicts an exemplary entry 410 of an AVM database, such as the database 250 of FIG. 2. As depicted in FIG. 4, the exemplary entry 410 contains a variety of fields, each of which can have use in executing a query/search of real estate properties. For example, a first useful field can be a “property identifier”, which can be a unique code associated with the property at interest. Other related fields can be the address of the property (which may also serve as a property identifier) and various geographic identifiers, which can serve to provide exact geographic information (latitude and longitude), school district information, zip code, housing development information, zoning information or any other information having geographic significance. Such fields, which can define a wide range of geographic regions can add value to a database by enabling unique geographic searches.

A second group of fields include an AVM value field, the date that the AVM value was calculated and various AVM confidence identifiers, which can provide a measure of confidence as to the accuracy of the AVM value. Such AVM confidence identifiers can include a number of similar properties on the market that have recently sold, a number of similar properties upon which an AVM value was determined and so on. Such AVM confidence identifiers may also consist of a set of one or more codes or values (e.g., a statistical variance) reflecting the above-mentioned (or similar) data.

Other information of interest can include various sale information fields, such as indicators as to whether the property is presently offered for sale, the offering/sale price, the date/time on market, the method of sale (e.g., for sale by owner, bankruptcy sale), the sales agent if any and so on. Still other information can include detailed description information of the property, such as the number of bedrooms, total square feet etc.

Again returning to FIG. 1, it should be appreciated that in view of FIGS. 3 & 4 the query device 260 can perform AVM-based queries on any number of regions defined by attributes such as a state, a city, a set of nearby cities a county or parish, adjacent counties, a zip code, proximity to a resource (e.g., a beach) or proximity to a specific location (e.g., within a 4 block radius of a train/subway station), a school district, a particular neighborhood, adjacent neighborhoods or a set of neighborhoods, a geographic region governed by a particular government body, a set of geographic regions governed by different government bodies and so on.

It should also be appreciated that the query device might also perform queries based on a geographic region provided by a user, such as a hand-drawn region provided using a computer mouse and a computer-generated map backdrop or area defined by latitude and longitude coordinates.

Still further, it should be appreciated that the query device 260 can perform queries based on certain information regarding the character of the property, such as acreage, type of property, number of bedrooms, etc.

Using the available information provided by the AVM database 250 and the services provided by the query device 260, it should be apparent that users of the provider 130 can identify properties of interest in a large number of new and useful ways. While such novel functionality can be of consequence by itself, it should also be appreciated that such functionality can be augmented by new and useful displays and interfaces.

For instance, in various embodiments, a number of properties identified by the query device 260 can be provided to the display device 270 of FIG. 1. The display device 270, in turn, can provide a variety of useful displays to a terminal, such as a personal computer, to aid in the review of such properties. In a first embodiment, such a display can take the form of an ordered list, i.e., a literal display. However, even straightforward literal displays can be augmented by hyperlinks to detailed descriptions of respective properties, to maps, to driving directions, to sale histories of the property or comparable/neighboring properties etc.

In addition to literal displays, electronic and printable maps (graphic displays) of one, some or all identified properties can be generated with superimposed icons or with other identifiers representing the identified properties. Such displays can also be augmented by interactive display tools. For example, in a particular embodiment, the display device 270 can provide a “pop up” window to a terminal in order to provide information of interest. For instance, in various embodiments a user accessing the provider 130 via a PC can “click” on a property icon embedded in an electronic map displayed on his screen. In response, the display device 270 can provide a window containing useful information, such as address, sale price and AVM data.

In still other embodiments, the display device 270 can provide specialized displays and interfaces for use in mobile terminals such that the provider 130 can provide interactive driving directions, which may be especially useful for users having a GPS device integrated into their terminal. Alternatively, the display device may provide comparable audio information including addresses, driving instructions etc, or information that might be displayed on the relatively tiny displays found on many mobile phones and PDAs.

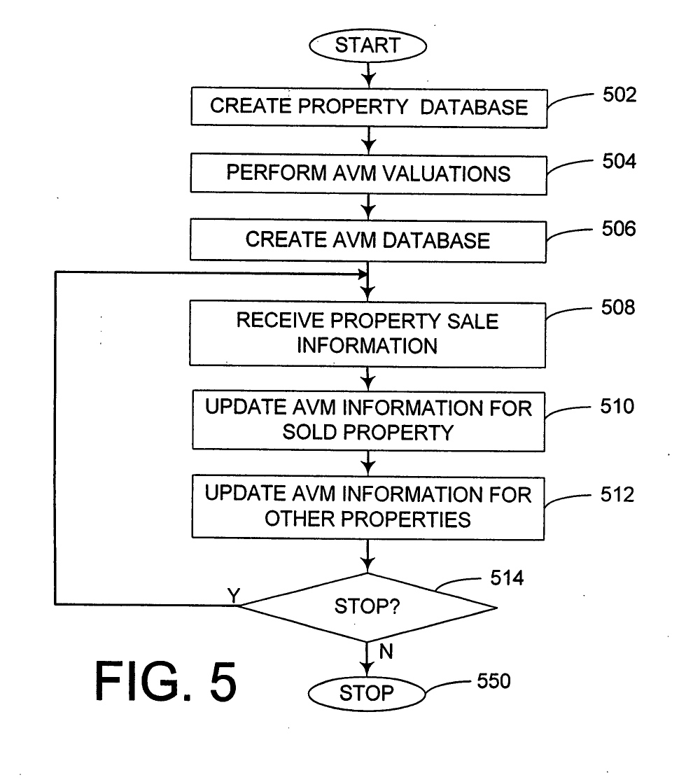

FIG. 5 is a flowchart outlining an exemplary operation according to the present disclosure for first creating an AVM-database, then updating the database to reflect changes in a particular market that may occur with each property sale. The process starts in step 502 where a database containing information about all, or substantially all, known properties in a particular geographic region of interest is created.

While the database of step 502 contains information about all known properties within a geographic region, it should be appreciated that, on other embodiments, the AVM database can be limited to only properties offered for sale, only properties sold in a particular fashion, e.g., for sale by owner or bankruptcy sale, properties of a particular type (e.g., townhouses), existing (not new) properties, properties proffered by a particular developer or builder, etc.

Still further, while the exemplary database contains only residential properties, it should be appreciated that the property database (and respective queries) can be expanded or changed to include any combination of undeveloped land, commercial properties, residential properties and so on. Control continues to step 504.

In step 504, AVM valuations are performed for each property of the database of step 502. In the exemplary step, AVM valuations can include AVM values, the date that the AVM value was determined and optionally include AVM confidence indicators. Next, in step 506, an AVM database is created using the database of step 502 and the AVM information produced in step 504. Control continues to step 508.

In step 508, sale information relating to a particular property is received. Next, in step 510, the AVM database of step 506 can be updated to reflect the new market value of the sold property based on the sale price. In various embodiments, the new AVM value can be the sale value. However, in view that circumstances occur where the sale value of the property may not reflect the actual market value, it should be appreciated that a new AVM value might be generated and the AVM database updated appropriately. Still further, the AVM value can be modified based on other financial considerations, e.g., points paid for by the seller and so on. Control continues to step 510.

In step 510, the AVM database of step 506 can be further updated to reflect the new market value of other properties based on the sale price, an adjusted sale price (i.e., the sale price adjusted based on concessions made by the buyer or seller (or other value) that might better reflect the actual sale value of the property) and/or modified AVM value of the sold property. As discussed above, such properties to be updated may include only properties in a same development, properties within a same locality, properties within a certain distance from the sold property, properties having similar structural features, such as acreage, area, builder, number of bedrooms etc.

Still further, AVM property adjustments can be “weighted” according to structural, geographical, spatial (distance and/or vector) and other differences. For example, if two properties are identical to a third sold property, the AVM valuation updates for each of the two may nonetheless be different if one property is closer to the sold property than the other. That is, AVM valuation can be weighted as a function of geographic proximity. Control continues to step 512.

In step 514, a determination is made as to whether to further modify the database. If the database is to be further modified, control jumps back to step 508 where new sale information is received; otherwise, control continues to step 550 where the process stops.

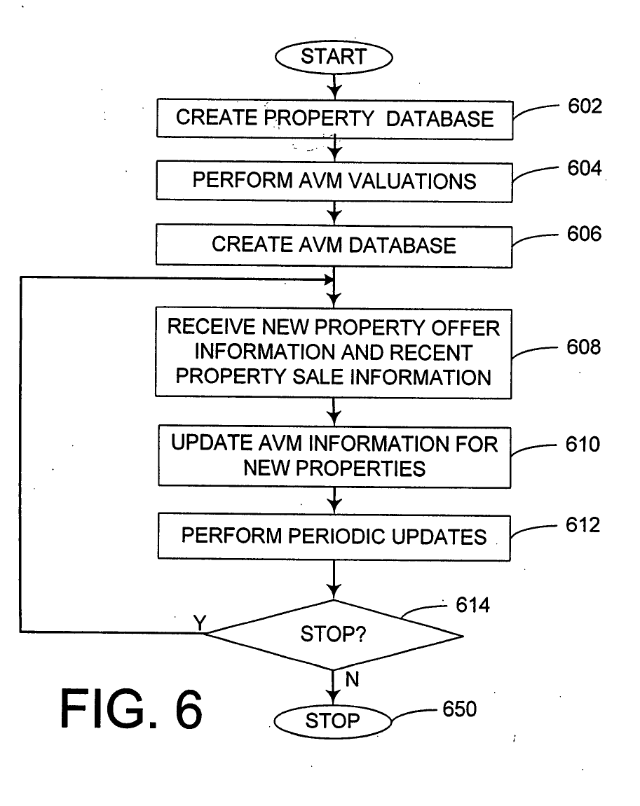

FIG. 6 is a flowchart outlining a second exemplary operation according to the present disclosure for first creating an AVM-database, then updating the database to reflect changes in a particular market that may occur with each property sale. The process starts with steps 602-606, which are substantially similar to steps 502-506 discussed above, with control progressing to step 608.

In step 608, information relating to both new properties offered for sale and sale information relating to recently sold properties is received. Next, in step 610, the AVM database of step 606 can be updated to reflect the new market value of the both new properties offered for sale and the sold properties. Then, in step 612, the AVM database of step 506 can be periodically updated according to any of the techniques described above. Control continues to step 614

In step 614, a determination is made as to whether to further modify the database. If the database is to be further modified, control jumps back to step 608 where new sale information is received; otherwise, control continues to step 650 where the process stops.

In various embodiments where the above-described systems and/or methods are implemented using a programmable device, such as a computer-based system or programmable logic, it should be appreciated that the above-described systems and methods can be implemented using any of various known or later developed programming languages, such as “C”, “C++”, “FORTRAN”, Pascal”, “VHDL” and the like.

Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform the above-described systems and/or methods.

For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods related to AVM-related services.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A storage medium containing a database of property related information that, when accessed by a computer, can enable a user to perform a number of property-related queries, the database comprising:
records on a substantial portion of every residential property in a defined geographic region that is offered for sale, wherein each entry of the property database includes,
an identifying field identifying a specific property, and
an automated valuation field containing an AVM produced value of the identified property.

2. The storage medium of claim 1, wherein the storage medium includes records on a majority of every residential property in a defined geographic region that is offered for sale, wherein each entry of the property database includes,

3. The storage medium of claim 2, wherein the storage medium includes records on substantially all residential properties in a defined geographic region that are offered for sale, wherein each entry of the property database includes,

4. The storage medium of claim 2, wherein at least one AVM value is produced using relative spatial information having a resolution of at least one-hundred meters.

5. The storage medium of claim 4, wherein at least one AVM value is produced using relative spatial information having a resolution of at least ten meters, and wherein the spatial information includes position data of at least two properties produced by a survey.

6. The storage medium of claim 4, wherein the position data of the properties is derived using either of a GPS survey or a GIS.

7. The storage medium of claim 1, wherein the defined geographic region includes one of (1) at least two adjacent counties or parishes, (2) at least 25% of a state or province, or (3) at least two neighboring cities.

8. The storage medium of claim 7, wherein the database further includes indicators indicating substantially all known properties that are offered for sale within the defined geographic region.

9. The storage medium of claim 8 offering wherein the database further includes a price for each property offered for sale within the defined geographic region.

10. An apparatus for creating and maintaining a database containing information relating to residential properties, comprising:
an AVM device configured to receive information relating to at least a majority of every known property within a defined geographical region that is currently offered for sale;
wherein the AVM device is configured to perform an AVM operation on each property to produce an AVM value for each property; and
wherein the AVM device is further configured to create an AVM database containing entries of a plurality of the AVM values.

11. The apparatus of claim 10, wherein the AVM device is further configured to create an AVM database containing entries of substantially all of the AVM values.

12. The apparatus of claim 11, wherein the AVM device is further configured to create an AVM database containing entries of all of the AVM values.

13. The apparatus of claim 10, wherein the AVM device is also configured property to produce one or more AVM confidence indicators, then store the one or more AVM confidence indicators in the AVM database.

14. The apparatus of claim 10, wherein an AVM device configured to receive information relating to substantially every known property within the defined geographical region, produce an AVM value for each property and store the produced AVM values in the AVM database.

15. An apparatus for creating and maintaining a database containing information relating to residential properties, comprising:
an AVM device configured to receive information relating to a residential property offered for sale in a defined geographical region, the first property having a first offering price;
wherein the AVM device is configured to perform an AVM operation on the first property to produce a first AVM value for the first property;
wherein the AVM device is further configured to update the AVM database using the first AVM value; and
where the AVM database contains entries of a plurality of the AVM values relating to other properties within the geographical region.

16. The apparatus of claim 15, where the AVM database contains entries of AVM values relating to substantially all residential properties within the geographical region.

17. The apparatus of claim 16, wherein the AVM device is further configured to replace an existing AVM entry with the first AVM entry.

18. The apparatus of claim 16, wherein the AVM device is further configured to update one or more second properties that are identified to be affected by the sale of the first property.

19. The apparatus of claim 18, wherein the AVM device configured to update the one or more second properties in response to the sale of the first property.

20. The apparatus of claim 18, wherein at least one of the one or more second properties includes a neighboring property.

21. The apparatus of claim 18, wherein at least one of the one or more second properties includes a property having comparable structural features.

22. The apparatus of claim 15, wherein the AVM device is configured to automatically update properties on a periodic basis.

23. The apparatus of claim 22, wherein the AVM device is configured to automatically update the AVM value for an individual property on a periodic basis.

24. The apparatus of claim 22, wherein the AVM device is configured to automatically update the AVM values for regions of properties on a periodic basis.

25. The apparatus of claim 22, wherein the AVM device is configured to update AVM values for regions of properties based on a user command.

26. The apparatus of claim 22, wherein the AVM device is configured to update the AVM value for an individual property based on a user command.

27. A storage medium containing sets of instructions that, when accessed by a computer, can enable a user to perform a number of property-related queries, the storage medium comprising:
a first set of instructions for receiving records on substantially every residential property in a defined geographic region offered for sale; and
a second set of instructions for producing an AVM value for each property for which records are received.

28. The storage medium of claim 27, further comprising:
a third set of instructions for entering each produced AVM value in an AVM database.

29. The storage medium of claim 28, where the AVM database contains entries of AVM values relating to substantially all residential properties within the geographical region.

30. A storage medium containing sets of instructions that, when accessed by a computer, can enable a user to perform a number of property-related queries, the storage medium comprising:
a first set of instructions for updating an AVM database, including updating a first AVM value for at least one residential property offered for sale in a defined geographic region.

31. The storage medium of claim 30, further comprising a second set of instructions for receiving information relating to a sale of a second residential property within defined the geographic region, and wherein the first set of instructions are configured to update AVM values based upon a sale price of the second property.

32. The storage medium of claim 31, wherein the first set of instructions are configured to update the AVM values based upon value received for the second property not reflected in the sale price.

33. The storage medium of claim 31, wherein the first set of instructions are configured to automatically update AVM values automatically on a periodic basis.

34. The storage medium of claim 33, wherein the first set of instructions are configured to update AVM values for individual properties automatically on a periodic basis.

35. The storage medium of claim 33, wherein the first set of instructions are configured to update AVM values for regions of properties on a periodic basis.

36. The storage medium of claim 30, wherein the first set of instructions are configured to update AVM values for regions of properties based on a user command.

37. The storage medium of claim 30, wherein the first set of instructions are configured to update an AVM value for an individual property based on a user command.
